(19) United States
(12) Patent Application Publication
(10) Pub. No.: US 2008/0091196 A1
(43) Pub. Date: Apr. 17, 2008

(54) WIRE-GUIDED ASPIRATION NEEDLE

(75) Inventor: STEPHEN E. DEAL, Charlotte, NC (US)

Correspondence Address: BRINKS HOFER GILSON & LIONE/CHICAGO/COOK, PO BOX 10395, CHICAGO, IL, 60610, US

(73) Assignee: WILSON-COOK MEDICAL INC., Winston-Salem, NC (US)

(21) Appl. No.: 11/869,248

(22) Filed: Oct. 9, 2007

Related U.S. Application Data
(60) Provisional application No. 60/852,197, filed on Oct. 17, 2006.

(51) Int. Cl. A61B 18/14

(52) U.S. Cl. 606/45

(57) ABSTRACT

A needle knife sphincterotome comprising a tensioning filament for positionally adjusting its distal end and a needle knife configured for tissue aspiration. The sphincterotome includes an actuatable handle attached to an elongate shaft having a needle knife near its distal end. The tensioning filament includes proximal and distal attachments and is configured such that actuation of the handle introduces a tension in the tensioning filament that bends, arches, or otherwise deforms the distal end of the sphincterotome shaft.

### CROSS-REFERENCE TO RELATED APPLICATIONS

This application claims priority to U.S. Provisional Application Ser. No. 60/852,197, filed Oct. 17, 2006, which is incorporated herein by reference in its entirety.

### FIELD OF THE INVENTION

The present device relates to medical devices, and specifically to improvements to a wire-guided aspiration needle or needle knife.

### BACKGROUND OF THE INVENTION

In endoscopic, or other minimally invasive surgery, generically referred to herein as endoscopic surgery, a needle knife may be used in conjunction with an endoscope to provide surgical cutting inside a patient. For example, a needle knife may used during certain procedures to make an incision in a sphincter (sphincterotomy). As one example, a common treatment of cholecystitis includes the removal of gallstones from the common bile duct. This is frequently done endoscopically with the use of a duodenoscope. The common bile duct proceeds from the junction of the common hepatic duct with the cystic duct, which is open to the gall bladder, and merges with the pancreatic duct, forming the ampulla of Vater, which itself opens into the duodenum at the papilla of Vater. The sphincter of Oddi is a muscular ring that controls passage of fluid from the ampulla of Vater into the duodenum. For removal of gallstones in an endoscopic procedure, access to the common bile duct for removal of gallstones is eased using a needle knife sphincterotome (or side-wire sphincterotome) to incise or sever the sphincter of Oddi. The needle knife sphincterotome is introduced through the duodenoscope and guided through the duodenum to the common bile duct. Once the tool is guided into the sphincter, its cutting element is used to incise the sphincter, and thereby improve access to the bile duct and impacted gallstones. The needle knife cutting tip may be hollow and provide a path of fluid communication with a lumen from the distal cutting element through an elongate catheter forming the major length of its body to allow for aspiration of tissue therethrough.

Another example of a common procedure utilizing a needle knife sphincterotome is endoscopic retrograde cholangiopancreatography (ERCP), a diagnostic visualization technique used for variety of clinical applications. In this procedure, a contrast fluid such as a radio-opaque dye is introduced through a tube into the ampulla of Vater. A needle knife sphincterotome is often employed to provide access through the sphincter of Oddi in the same manner as described above. ERCP is often used in diagnosis of cholecystitis, as well as in the diagnosis and treatment of other conditions of the pancreatic and common bile ducts and related structures.

As illustrated in FIG. 1, one prior art needle knife sphincterotome 100 includes a handle 110 attached to a catheter shaft 102 generally made of PTFE (polytetrafluoroethylene) or another flexible material. The sphincterotome 100 includes a wire guide lumen 106 extending through the shaft 102 and open to a side port 104, which allows the tome 100 to be directed over a wire guide 108 in either of a long-wire (not shown) or short-wire (“rapid exchange”) manner, a configuration known in the art as “convertible” or “dual use”. An electrode means 114 is included in the handle 110 for connection to an electrosurgical generator (not shown) for providing current to a distal needle knife 109. Electric current passed from an electrode 114 in the handle 110 enables the knife 109 to act as an electrosurgical cutting element that may be used effectively to cut and cauterize tissue, such as the sphincter of Oddi in the exemplary procedures described above. The knife 109 may include a lumen (not shown) extending therethrough (and extending up through the shaft) to aspirate tissue.

It would be advantageous to provide a distal steering means enabling a user to direct the distal end of a needle knife sphincterotome by manipulation of a proximal handle means independent of a wire guide.

### BRIEF SUMMARY OF THE INVENTION

In one aspect, the present invention includes a sphincterotome that includes a handle assembly, a flexible elongate shaft, and a tensioning filament. The flexible elongate shaft is connected to the handle assembly and includes a first lumen, a proximal end, and a distal end. An electroconductive component extends through at least a portion of the elongate shaft and includes a proximal component section and a distal component section. The proximal component section is attached to an electrode in the handle assembly, and the distal component section extends beyond the distal end of the elongate shaft to form a cutting element. The tensioning filament includes a proximal filament section and a distal filament section. The proximal filament section is slidably disposed at least partially in the first lumen and has a proximal attachment to the handle assembly. A portion of the distal filament section exits the first lumen and is attached near the distal end of the shaft. The attachment mounting of the filament is configured such that, when the handle assembly is actuated, the filament is moved longitudinally in a manner that deforms a distal portion of the shaft.

In another aspect, the present invention includes a needle knife sphincterotome that includes a proximal handle assembly, an elongate shaft distally extendable from the handle assembly, an electroconductive needle knife element disposed in the elongate shaft and projecting generally distally therefrom, and a tensioning filament connecting the handle assembly to a region near a distal end of the elongate shaft and configured such that an actuation of the handle assembly exerts a deforming tension upon the distal end of the elongate shaft.

### BRIEF DESCRIPTION OF THE DRAWINGS

FIG. 1 is a typical prior art sphincterotome;

FIG. 2 is an external side view of a sphincterotome embodiment of the present invention;

FIG. 2A is a cross-sectional view of the embodiment of FIG. 2, taken along line 2A-2A;

FIGS. 2B-2B are side views of the embodiment of FIG. 2, showing—respectively—default/relaxed and actuated states of the device;

FIG. 3 shows an external side view of another sphincterotome embodiment of the present invention in a non-actuated state;

FIGS. 3A-3B depict a handle-locking mechanism of the embodiment shown in FIG. 3;

FIG. 3C illustrates the sphincterotome of FIG. 3 in an actuated state; and

FIG. 3D shows a cross-section of a shaft portion of the sphincterotome of FIG. 3 taken along line 3D-3D.

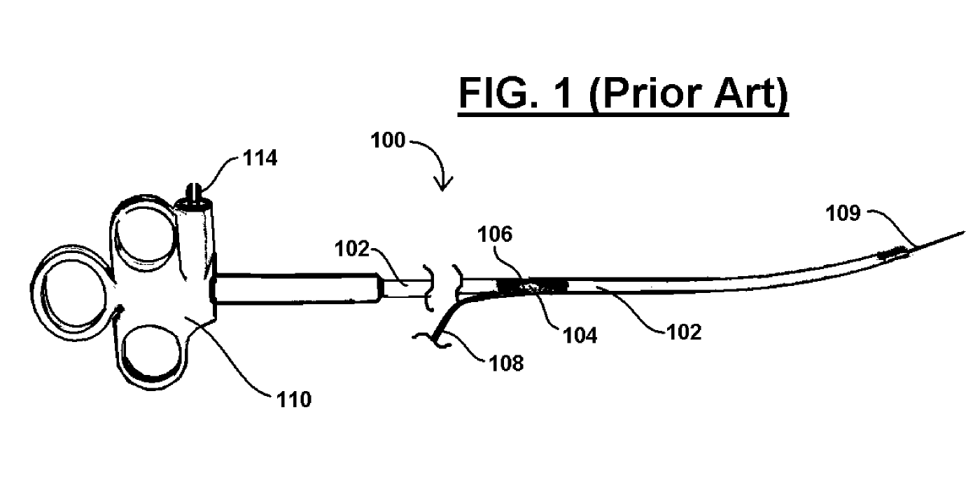

### DETAILED DESCRIPTION

The following disclosure describes embodiments of a needle knife sphincterotome including a tensioning filament/wire that provides for steering the distal end of the sphincterotome independent of a wire guide. Those of skill in the art will appreciate that variations of the described embodiments as well as other embodiments are possible and are within the scope of this application.

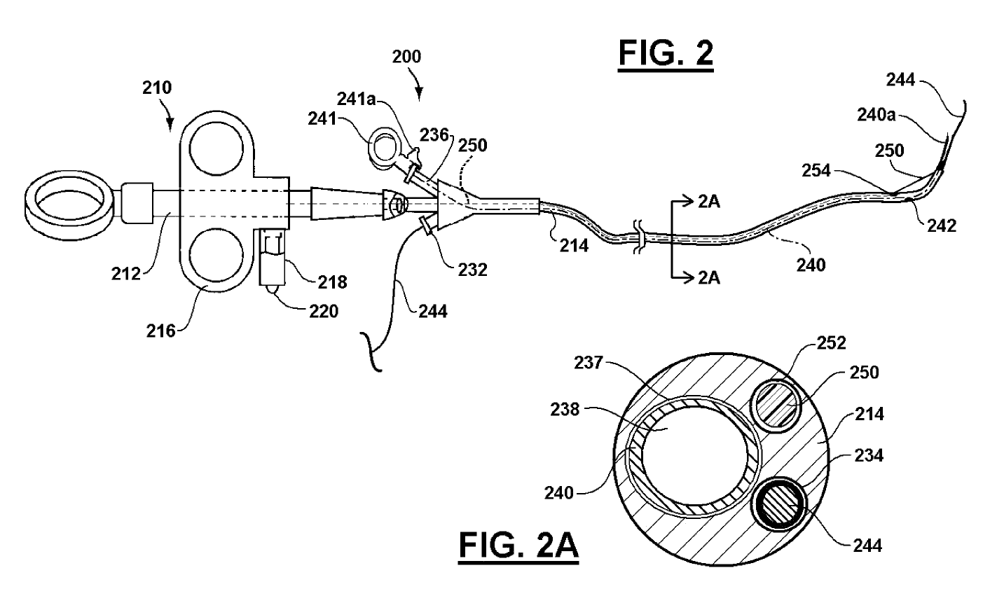

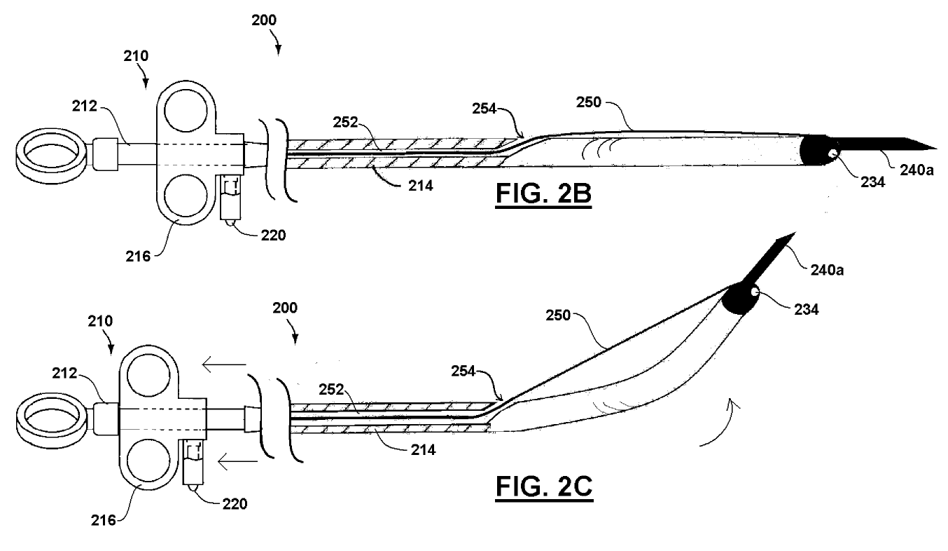

FIGS. 2-2A illustrate one embodiment of a needle knife sphincterotome 200. FIG. 2 shows a side external view of the tome 200, and FIG. 2A shows a cross-sectional view along the perspective of line 2A-2A. The proximal end includes a handle assembly 210, which has a first portion and a second portion. In the illustrated embodiment, the first portion is a one-ring (also referred to as a thumb ring or stem) handle component 212 that is attached to the proximal end of the catheter shaft 214 of the sphincterotome 200. The second portion is a two-ring (also referred to as a finger ring or spool) handle component 216 that is slidably mounted to the one-ring handle component 212. The two-ring handle component 216 is connected to a steering filament 250. The two-ring handle component 216 includes a housing 218 supporting an electrode 220. Also, the two-ring handle component includes a dual port structure 230, which has a first port 232 open to a wire guide lumen 234 of the catheter and a second port 236 that provides access to a needle lumen 237 and, optionally, to an aspiration/irrigation lumen 238 that extends through an aspiration needle/needle knife 240, which itself is slidably disposed through the needle lumen 237. Those of skill in the art will appreciate that the lumen 238 may be configured and used for aspiration of tissue from the distal to the proximal portion of the needle 240, and/or for introduction of a fluid therethrough (e.g., irrigation fluid, radio-contrast fluid). A body portion of the knife 240 extends proximally and is in electroconductive contact with the electrode 220. In other embodiments, the needle 240 may be solid (i.e., lacking a lumen). Persons of skill in the art will appreciate that many other embodiments of a sphincterotome handle are possible and practicable within the scope of the present invention. For example, the handle assembly may use a different number of rings, a trigger grip, or other gripping surfaces adapted for manipulating a sphincterotome. As another example, the connection between handle components and the shaft and filament may be reversed. Other structures such as a utility port for fluid communication access to a lumen in the sphincterotome shaft may also be located on a first or second portion of a handle assembly.

In the illustrated embodiment, the catheter shaft 214 includes a side port 242 near the distal end, which provides for use of the tome 200 along a wire guide in “rapid exchange configuration” by allowing a wire guide to pass out through the side port 242 without having to travel through the length of the shaft 214. As shown, a wire guide 244 extends through the wire guide lumen 234 in a “long wire configuration.” During a procedure using the tome 200, it is preferable that the wire guide 244 not extend beyond a distal cutting/coagulation end 240a of the knife 240, which end 240a may be disposed in line with, parallel to, or at an angle to a central longitudinal axis of the shaft 214. In the illustrated embodiment, the needle 240 includes a proximally-mounted guide-ring 241, which allows the needle end 240a to be retracted into or extended from the needle lumen 237 of the shaft 214. The guide-ring 241 includes an optional catch 241a for engaging the port 236 and holding the needle 240 in the extended position illustrated in FIG. 2. In other embodiments, the needle 240 may be fixed in an extended position.

The tome 200 also includes the distal-steering filament 250. The proximal end of the filament 250 is attached to the two-ring handle component 216, and the filament 250 extends distally through a filament lumen 252 to and through a side aperture 254 near the distal catheter end, and is attached nearer the distal end of the catheter 214. The filament 250 provides a user with the ability to steer the distal end of the tome catheter 214 by moving the handle components 212, 216 relative to each other. As shown in FIG. 2B, which shows the handles and includes a detail, partially cut-away view of a distal end of the sphincterotome 200, the distal end portion of the catheter 214 is generally aligned with its main longitudinal axis, which is the default position for the catheter 214 and handles. FIG. 2C illustrates the same perspective view as FIG. 2B and shows that, when the handle components 214, 216 are drawn together, the filament 250 is pulled proximally relative to the catheter 214, which bends, curves, or otherwise deforms the distal catheter end as the filament 250 is drawn proximally through the side aperture 254 and its distal end pulls on the distal catheter end. During an operation of the tome 200, this allows a user greater steering control of the distal catheter end and the knife end 240a mounted thereon, specifically allowing the user to move the axis of the knife end 240a out of alignment with the primary longitudinal axis of the catheter 214. This feature may allow for steering around corners, as well as allowing the knife end 240a to be used on a surface that is parallel (or nearly so) with the primary longitudinal axis of the tome's catheter 214. In an alternative embodiment, the filament 250 may be electroconductive and be in electroconductive communication with the electrode 220 such that the filament may be used as a cutting/coagulation wire in the same manner as side-wire sphincterotomes known in the art (e.g., Fusion® OMNI-Tome by Cook Endoscopy).

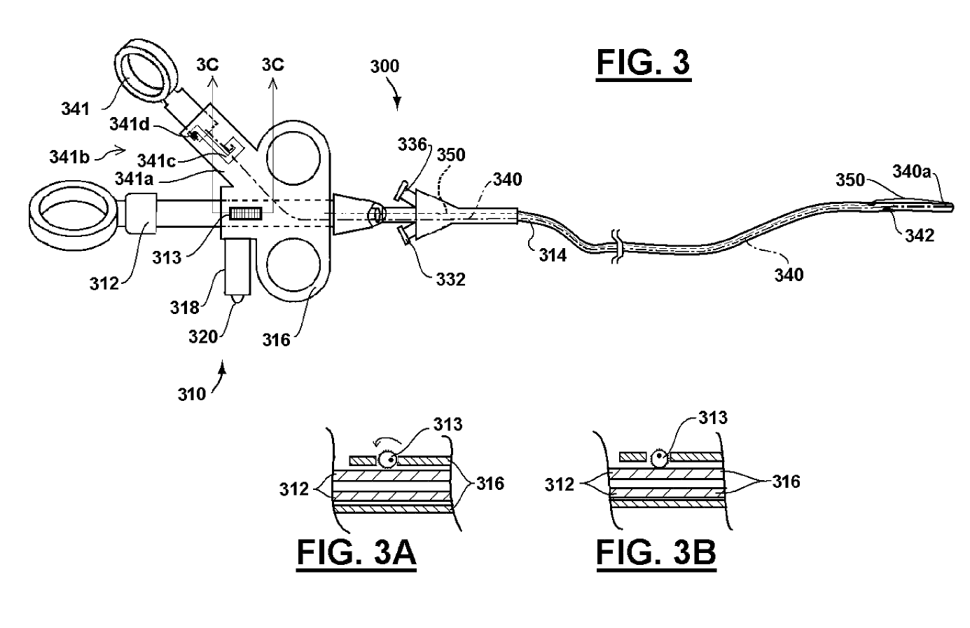

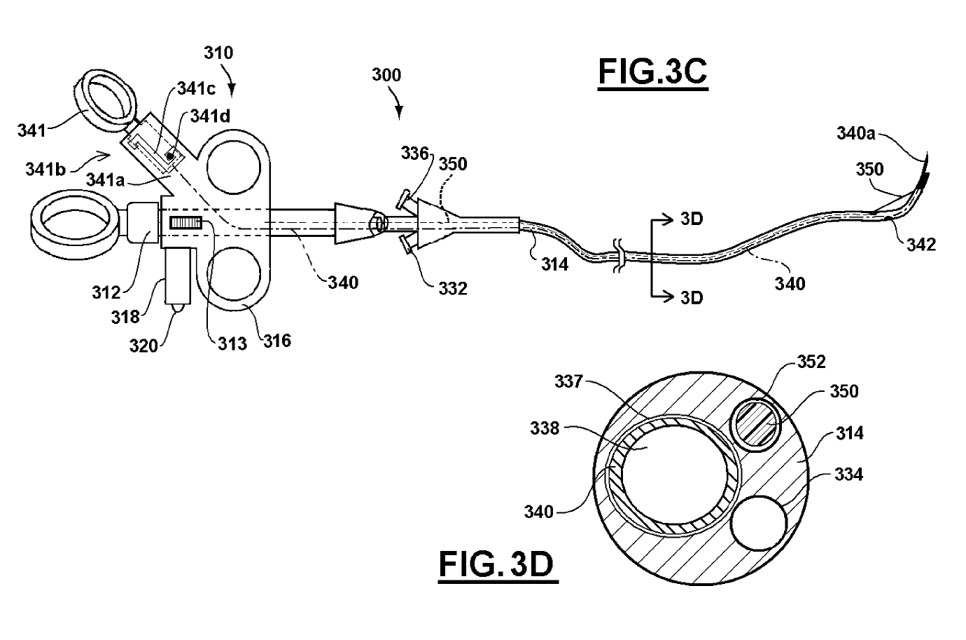

FIGS. 3-3D illustrate another embodiment of a needle knife sphincterotome 300 configured with a handle-locking mechanism, a needle actuation mechanism, and a means for distorting a distal portion of the tome 300. FIG. 3 shows a side external view of the tome 300 in a non-deployed state. The proximal end includes a handle assembly 310, which has a first portion and a second portion. In the illustrated embodiment, the first portion is a one-ring (also referred to as a thumb ring or stem) handle component 312 that is attached to the proximal end of the catheter shaft 314 of the sphincterotome 300. The second portion is a two-ring (also referred to as a finger ring or spool) handle component 316 that is slidably mounted to the one-ring handle component 312. The two-ring handle component 316 is connected to a steering filament 350. The two-ring handle component 316 includes a housing 318 supporting an electrode 320.

The handle assembly 310 has a locking mechanism that includes an off-center-mounted friction wheel 313 rotatably mounted to the two-ring handle component 316 and disposed such that its surface may frictionally engage the one-ring handle component 312 to limit the ability of, or prevent, the handle components 312, 316 from sliding relative to each other. FIGS. 3A-3B show a diagrammatic cross-section along line 3A-3A. Specifically, FIG. 3A depicts the friction wheel 313 in an “unlocked orientation” wherein the two handle portions 312, 316 are freely slidable relative to each other. In contrast, FIG. 3B shows the friction wheel 313 in a “locked orientation” where it is rotated to frictionally engage a surface of the one-ring handle component 312. The engagement shown is most effective at preventing movement of the handles 312, 316 apart from each other, and those of skill in the art will appreciate that other mechanisms for locking the handles relative to each other may be provided in accord with the present invention. For example, a transversely mounted thumb-screw, a detent mechanism, or some other locking means may be provided.

Also, the two-ring handle component includes a distal dual port structure 330, which has a first port 332 open to a wire guide lumen 334 of the catheter and a second port 336 that optionally provides access to a needle lumen 337 and/or to an aspiration/irrigation lumen 338 that extends through an aspiration needle/needle knife 340, which itself is slidably disposed through the needle lumen 337. In FIG. 3, the needle 340 is shown in a retracted position Those of skill in the art will appreciate that the lumen 338 may be configured and used for aspiration of tissue from the distal to the proximal portion of the needle 340, and/or for introduction of a fluid therethrough (e.g., irrigation fluid, radio-contrast fluid). A body portion of the knife 340 extends proximally and is in electroconductive contact (not shown) with the electrode 320. In other embodiments, the needle 340 may be generally solid (i.e., lacking a lumen). Persons of skill in the art will appreciate that many other embodiments of a sphincterotome handle are possible and practicable. For example, the handle assembly may use a different number of rings, a trigger grip, or other gripping surfaces adapted for manipulating a sphincterotome. As another example, the connection between handle components and the shaft and filament may be reversed. Other structures such as a utility port for fluid communication access to a lumen in the sphincterotome shaft may also be located on a first or second portion of a handle assembly.

As shown in FIGS. 3 and 3C, the needle 340 includes a proximally-mounted guide-ring 341, which allows the needle end 340a to be retracted into or extended from the needle lumen 337 of the shaft 314. The guide-ring 341 is mounted to a housing 341a, which includes an optional catch 341b configured for holding the needle 340 in a retracted position as shown in FIG. 3, or in an extended position as illustrated in FIG. 3C. The optional catch 341b is configured as a bayonet-type slide catch and includes a slot 341c in which travels a boss 341d protruding from the guide-ring 341. As those of skill in the art will appreciate from the depictions in FIGS. 3 and 3C, the needle end 340a can be extended distally by actuation of the guide-ring 341. Actuation of the guide-ring includes rotating the guide-ring 341 to guide its boss 341d out of an upper inverted-J-portion of the slot 341c, then moving the guide-ring 341 down/distally so that the boss 341d travels down the major length of the slot 341c. The actuation is concluded by rotating the guide-ring 341 to guide the boss 341d into engagement with a lower J-portion of the slot 341c.

In the illustrated embodiment, the catheter shaft 314 includes a side port 342 near the distal end, which provides for use of the tome 300 along a wire guide in “rapid exchange configuration” by allowing a wire guide to pass out through the side port 342 without having to travel through the length of the shaft 314. During a procedure using the tome 300, it is preferable that the wire guide (not shown) not extend beyond a distal cutting/coagulation end 340a of the knife 340, which end 340a may be disposed in line with, parallel to, or at an angle to a central longitudinal axis of the shaft 314.

The tome 300 also includes the distal-steering filament 350. The proximal end of the filament 350 is attached to the two-ring handle component 316, and the filament 350 extends distally through a filament lumen 352 to and through a side aperture 354 near the distal catheter end, and is attached nearer the distal end of the catheter 314. The filament 350 provides a user with the ability to steer the distal end of the tome catheter 314 by moving the handle components 312, 316 relative to each other. FIG. 3C illustrates the same view as FIG. 3A and—in addition to the extension of the needle end 340a described above—shows that, when the handle components 314, 316 are drawn together, the filament 350 is pulled proximally relative to the catheter 314, which bends, curves, or otherwise deforms the distal catheter end as the filament is drawn proximally through the side aperture 354 and its distal end pulls on the distal catheter end. During an operation of the tome 300, this allows a user greater steering control of the distal catheter end (independent of the wire guide, for example, if the wire guide has been partially retracted) and the knife end 340a mounted thereon, specifically allowing the user to move the axis of the knife end 340a out of alignment with the primary longitudinal axis of the catheter 314. This feature may allow for the knife end 340a to be used on a surface that is parallel (or nearly so) with the primary longitudinal axis of the tome's catheter 314. As shown in the illustrated embodiment, actuation of the needle end 340a and the filament 350 are independent of each other. FIG. 3D shows a cross-sectional view of the catheter portion 314 along line 3D-3D.

In preferred embodiments, one or more optically visible and/or radio-opaque markers (not shown) are placed at predetermined locations in or on the catheter to aid fluoroscopic imaging and navigation during procedures using a device of the present invention.

Persons of skill in the art will appreciate that variants of the sphincterotome with a steering mechanism described herein are within the scope of the present invention. It is therefore intended that the foregoing detailed description be regarded as illustrative rather than limiting, and that it be understood that it is the following claims, including all equivalents, that are intended to define the spirit and scope of this invention.

I claim:

1. A sphincterotome, comprising:
a handle assembly;
a flexible elongate shaft connected to the handle assembly and comprising a first lumen, a proximal end, and a distal end;
an electroconductive component extending through at least a portion of the elongate shaft and comprising a proximal component section and a distal component section, the proximal component section having a proximal attachment to an electrode, and the distal component section extending beyond the distal end of the elongate shaft to form a cutting element; and
a tensioning filament comprising a proximal filament section and a distal filament section,
the proximal filament section being slidably disposed at least partially in the first lumen and having a proximal attachment to the handle assembly,
a portion of the distal filament section exiting the first lumen and having a distal attachment near the distal end of the shaft,
said filament configured such that an actuation motion of the handle assembly moves the filament longitudinally in a manner that deforms a distal portion of the shaft.

2. The sphincterotome of claim 1, wherein the electroconductive component comprises a longitudinal lumen configured for aspiration of tissue therethrough.

3. The sphincterotome of claim 1, wherein the tensioning filament is electroconductive, and the proximal filament portion is in electroconductive contact with the electrode.

4. The sphincterotome of claim 1, wherein the handle assembly comprises:
a first ring handle structure connected to the elongate shaft;
a second ring handle structure mounted to the first ring handle structure in a manner allowing proximal and distal sliding movement of the second ring handle structure relative to the first ring handle structure;
the second ring handle structure comprising the proximal attachment of the tensioning filament;
wherein a movement of the second ring handle structure relative to the first handle portion moves the filament longitudinally within the first lumen.

5. The sphincterotome of claim 4, further comprising a locking mechanism configured to limit movement of the first ring handle structure relative to the second ring handle structure.

6. The sphincterotome of claim 1, further comprising a wire guide lumen disposed longitudinally through at least a portion of the elongate shaft.

7. The sphincterotome of claim 6, wherein the elongate shaft comprises a side port that is disposed near the distal end and is open to the wire guide lumen.

8. The sphincterotome of claim 1, further comprising at least one radio-opaque marker.

9. The sphincterotome of claim 1, wherein the electroconductive component is configured such that its distal component section is at least partly retractable into the elongate shaft.

10. The sphincterotome of claim 9, comprising a generally proximal ring structure connected to, and configured for extending and retracting, the electroconductive component.

11. The sphincterotome of claim 10, wherein the generally proximal ring structure comprises a catch mechanism configured to substantially hold the electroconductive component in at least one of an extended or a retracted position.

12. A needle knife sphincterotome comprising:
a proximal handle assembly;
an elongate shaft extending distally from the handle assembly;
an electroconductive needle knife element disposed at least partially through the elongate shaft;
a tensioning filament connecting the handle assembly to a region near a distal end of the elongate shaft and configured such that an actuation of the handle assembly exerts a deforming tension upon the distal end of the elongate shaft.

13. The needle knife sphincterotome of claim 12, further comprising a wire guide lumen extending through at least a portion of the elongate shaft.

14. The needle knife sphincterotome of claim 12, wherein the electroconductive needle knife element is slidably disposed through at least a portion of a lumen of the elongate shaft and is configured to be distally extendable therefrom.

15. The sphincterotome of claim 12, further comprising a locking mechanism configured to limit movement of at least a portion of the handle assembly.

16. The sphincterotome of claim 12, further comprising a locking mechanism configured to limit movement of at least a portion of the electroconductive needle knife element.
